(19) United States
(12) Patent Application Publication
(10) Pub. No.: US 2025/0319110 A1
(43) Pub. Date: Oct. 16, 2025

(54) SURFACE CALR CHEMICAL INDUCERS

(71) Applicants: Leonard ZON, Brookline, MA (US); Cecilia Pessoa RODRIGUES, Cambridge, MA (US); THE CHILDREN'S MEDICAL CENTER CORPORATION, Boston, MA (US); PRESIDENT AND FELLOWS OF HARVARD COLLEGE, Cambridge, MA (US); HOWARD HUGHES MEDICAL INSTITUTE, Chevy Chase, MD (US)

(72) Inventors: Leonard ZON, Brookline, MA (US); Cecilia Pessoa RODRIGUES, Cambridge, MA (US)

(21) Appl. No.: 18/874,961

(22) PCT Filed: Jun. 7, 2023
(86) PCT No.: PCT/US2023/024646

§ 371 Date: Dec. 13, 2024

Related U.S. Application Data
(60) Provisional application No. 63/352,206, filed on Jun. 14, 2022.

(51) Int. Cl. A61K 31/706; A61K 31/53; A61K 31/635; A61K 45/06

(52) U.S. Cl. CPC A61K 31/706; A61K 31/53; A61K 31/635; A61K 45/06

(57) ABSTRACT

ROS signal upregulates surface CALR and promotes macrophage-HSC interactions, safeguarding the development of stem cells that are stressed or damaged. Described herein are methods of controlling hematopoiesis, e.g., reducing hematopoiesis and/or improving the quality control mechanisms of hematopoiesis, relating to the use or administration of at least one CALR agonist.

### CROSS-REFERENCE TO RELATED APPLICATIONS

This application claims benefit under 35 U.S.C. § 119(e) of U.S. Provisional Application No. 63/352,206 filed Jun. 14, 2022, the contents of which are incorporated herein by reference in their entirety.

### GOVERNMENT SUPPORT

This invention was made with government support under Grant No. HL131477 awarded by National Institutes of Health (NIH). The government has certain rights in the invention.

### TECHNICAL FIELD

The technology described herein relates to methods for controlling HSC proliferation and survival, e.g., to treat disorders in which HSC proliferation and survival is aberrant or nonoptimal.

### BACKGROUND

In many adult tissues, the maintenance of organ function depends on stem cells. Although there is a requirement of these for tissue repair, it is unknown which signals are quality assured during development. Macrophage-HSPC interactions during embryogenesis quality assure the nascent stem cell pool. Macrophages either fully engulf stem cells or partially eat them. In the latter situation, the stem cell goes on to divide. This is mediated by the “eat me” signal calreticulin (CALR) on the HSPC surface. Nonetheless, the signals triggering CALR expression on the HSPC surface mediating their removal or amplification remain unknown.

### SUMMARY OF THE INVENTION

Single-cell RNAseq analysis of the adult zebrafish marrow revealed a continuum of calr levels among the HSPC expression states, which correlated with the FoxO signaling, a pathway known to respond to reactive oxygen species (ROS). Probing surface CALR in human hematopoietic cell lines and zebrafish embryos, ROS scavengers lower surface CALR expression and macrophage interactions and FoxO CRISPR targeted embryos showed ROS accumulation associated with high surface CALR levels and decreased HSPC numbers. To systematically evaluate pathways triggering surface CALR, a panel of 1200 bioactive small molecules in human cells were screened. Surface CALR expression was evaluated by imaging a CALR-antibody coupled with a fluorophore and by a SPLIT-TURBO ID construct targeting the association of CHD2 (Cadherin 2), a membrane protein, and CALR. 93 out of 1200 compounds screened increased surface CALR with a robust dosage response. Chemical annotation further supported that ROS+ drugs were associated with FOXO1A and oxidative stress, while the ROS− drugs were associated with G protein-coupled receptor signaling and cellular calcium ion homeostasis. In vivo ROS− drugs induced macrophage-stem cell interaction and grooming behavior, while ROS+ drugs enhanced the macrophage-stem cell interaction, but not grooming. Collectively, the work described herein has identified ROS as a signal that upregulates surface CALR and promotes macrophage-stem cell interactions, safeguarding the development of stem cells that are stressed or damaged.

In one aspect of any of the embodiments, described herein is a method of reducing hematopoiesis in a subject in need thereof, the method comprising administering at least one CALR agonist to the subject. In one aspect of any of the embodiments, described herein is at least one CALR agonist for use in a method of reducing hematopoiesis in a subject in need thereof. In some embodiments of any of the aspects, the subject is a subject in need of treatment for clonal hematopoiesis, Clonal Hematopoiesis of Indeterminate Potential (CHIP), myelodysplastic syndrome (MDS), or leukemia. In some embodiments of any of the aspects, the hematopoiesis is pathological hematopoiesis.

In some embodiments of any of the aspects, the administration is oral. In some embodiments of any of the aspects, the administration is intravenous.

In some embodiments of any of the aspects, mitochondrial stimulation produces ROS. In some embodiments of any of the aspects, mitochondrial stimulation comprises mitochondrial modulation.

In some embodiments of any of the aspects, the at least one CALR agonist is at least one ROS− CALR agonist. In some embodiments of any of the aspects, the at least one ROS− CALR agonist is selected from the group consisting of: DL-threo-1-Phenyl-2-palmitoylamino-3-morpholino-1-propanol; cambinol; ganciclovir; 8-bromo-GMP; arvanil; thiocitrulline; and isoguvacine.

In some embodiments of any of the aspects, the at least one CALR agonist is at least one ROS+ CALR agonist. In some embodiments of any of the aspects, the at least one ROS+ CALR agonist is selected from the group consisting of: Ibudilast; zaprinast; FK-520; AG1478; bongkrekic acid; GW9508; fusidic acid; beta-lapachone; fusaric acid; fenspiride; zardaverine; docosahexaenoic acid, loxoprofen; AG1480; flufenamic acid; and MG-132.

In some embodiments of any of the aspects, the method further comprises administering to the subject at least one chemotherapeutic. In some embodiments of any of the aspects, the at least one chemotherapeutic is selected from 5-azacytidine; venetoclax; and guadecitabine.

In one aspect of any of the embodiments, described herein is a method of improving proliferation and/or survival of healthy hematopoietic stem cells, the method comprising contacting a population of hematopoietic stem cells (HSCs) with at least one CALR agonist. In one aspect of any of the embodiments, described herein is a ex vivo method of improving proliferation and/or survival of healthy hematopoietic stem cells, the method comprising contacting an ex vivo population of hematopoietic stem cells (HSCs) with at least one CALR agonist. In some embodiments of any of the aspects, the at least one CALR agonist is at least one ROS− CALR agonist. In some embodiments of any of the aspects, the at least one ROS− CALR agonist is selected from the group consisting of: DL-threo-1-Phenyl-2-palmitoylamino-3-morpholino-1-propanol; cambinol; ganciclovir; 8-bromo-GMP; arvanil; thiocitrulline; and isoguvacine.

In some embodiments of any of the aspects, the contacting occurs ex vivo. In some embodiments of any of the aspects, the population of HSCs and/or the progeny of the population of HSCs is subsequently administered to a subject.

In some embodiments of any of the aspects, the contacting comprises administering the at least one CALR agonist to a subject in need of improved proliferation and/or survival of healthy hematopoietic stem cells. In one aspect of any of the embodiments, described herein is at least one CALR agonist for use in a method of improving proliferation and/or survival of healthy hematopoietic stem cells in a subject.

In some embodiments of any of the aspects, the subject in need of improved proliferation and/or survival of healthy hematopoietic stem cells is a subject in need of or receiving a transplant or adoptive cell therapy. In some embodiments of any of the aspects, the administration is oral. In some embodiments of any of the aspects, the administration is intravenous.

### BRIEF DESCRIPTION OF THE DRAWINGS

FIGS. 1A-1B. FIG. 1A depicts single-cell RNAseq analysis of IRF8 KD zebrafish embryos, FIG. 1B depicts the ROS levels in CALR+ and CALR− blood progenitor cells.

FIG. 2 depicts the effect of ROS levels on surface CALR and stem cell-macrophage interactions.

FIGS. 3A-3B depict experiments in which Foxo3a perturbation was found to cause increased ROS, promote surface CALR and increase “dooming” events.

FIG. 4 depicts a schematic of a chemical screen to identify surface CALR inducers in a ROS-associated and a ROS-independent manner.

FIGS. 5A-5B depict validation of hits from the screen in vivo as CALR inducers.

FIGS. 6A-6C demonstrate that ROS− drugs tended to induce macrophage-stem cell interaction and proliferation while ROS+ drugs tended to promote dooming.

FIG. 7 depicts a graph of induction of surface CALR in vivo by the indicated compounds.

FIG. 8 demonstrates that only one of the compounds induced early apoptosis.

FIG. 9 demonstrates that the indicated compounds also induced surface CALR on human HSCs.

FIGS. 10A-10B demonstrate that ROS+ compounds were associated with a “dooming” fate and altered the mitochondrial potential.

FIGS. 11A-11B demonstrate that ROS+ compounds were associated with a “dooming” fate and altered the mitochondrial potential and morphology.

FIGS. 12A-12B demonstrate that the absence of HSPC-macrophage interaction caused the accumulation of mitochondrial superoxide.

FIGS. 13A-13B demonstrate that Foxo3a (F0) CRIPSANTS had lower clonal diversity.

FIGS. 14A-14B demonstrate that Foxo3a germlines reproduced the F0 phenotype.

FIG. 15 depicts the experimental design for a CRISPR-Cas9 screen to decode the molecular cues regulating surface CALR.

FIG. 16 depicts the experimental design for a CRISPR-Cas9 screen to decode the molecular cues regulating surface CALR.

FIG. 17 depicts a schematic of the pathways upregulated in the eat-me context.

FIGS. 18A-18D demonstrate that Asxl1-mutant progenitor cells showed higher engagement with macrophages.

FIGS. 19A-19C demonstrate that Asx1-mutant progenitor cells have higher surface CALR that can be modulated with the ROS/CALR inducers compounds.

### DETAILED DESCRIPTION

Calreticulin (CALR) moves to the cell surface of HSCs. Once present on the cell surface, CALR mediates interactions with macrophages, in a process that serves as a type of quality assurance of HSCs. Some HSCs are full engulfed by the macrophages and do not proliferate and differentiate. Another group of HSCs are only partially engulfed and do go on to proliferate and contribute to hematopoiesis.

The inventors have discovered that the movement of CALR to the HSC cell surface is promoted by FoxO signaling and, optionally ROS levels. CALR agonism in the presence of or via ROS induction tends to produce full engulfment by macrophages. CALR agonism in the absence of or independently of ROS induction tends to produce partial engulfment by macrophages, preceded by alteration in mitochondrial potential and the accumulation of superoxide in the mitochondria. Further, the inventors have discovered CALR agonists that operate by each of these mechanisms, thereby inducing quality assurance of HSCs, and the further ability to weight the quality assurance towards HSC killing or HSC proliferation.

Accordingly, in one aspect of any of the embodiments, described herein is a method of reducing hematopoiesis in a subject in need thereof, the method comprising administering at least one CALR agonist to the subject.

As used herein, the term “hematopoiesis” refers to the formation and development of blood cells. In the embryo and fetus, it takes place in a variety of sites including the liver, spleen, thymus, lymph nodes, and bone marrow; from birth throughout the rest of life it is mainly in the bone marrow with a small amount occurring in lymph nodes. A subject in need of a reduction in hematopoiesis can be a subject with higher than desired, or a pathological level of hematopoiesis. In some embodiments of any of the aspects, the subject is in need of treatment for pathological hematopoiesis. Non-limiting examples of conditions in which the level of hematopoiesis contributes to disease or disease progression include clonal hematopoiesis, Clonal Hematopoiesis of Indeterminate Potential (CHIP), myelodysplastic syndrome (MDS), and leukemia. In some embodiments of any of the aspects, the subject has, has been diagnosed with, or is in need of treatment for: clonal hematopoiesis, Clonal Hematopoiesis of Indeterminate Potential (CHIP), myelodysplastic syndrome (MDS), or leukemia.

As used herein, “CALR” or “calreticulin” refers to a chaperone protein found primarily in the endoplasmic reticulum. In hematopoietic stem cells, CALR can be found on the cell surface. The structure and sequence of CALR is known for a number of species, e.g, human CALR is found in the NCBI database at Gene ID 811.

As used herein, the term “agonist” refers to an agent which increases the expression and/or cell surface (e.g., HSC cell surface) levels of CALR by at least 10% or more, e.g. by 10% or more, 50% or more, 100% or more, 200% or more, 500% or more, or 1000% or more. The efficacy of an agonist, e.g. its ability to increase the expression and/or level of CALR can be determined, e.g. by measuring the level of CALR. Methods for measuring the level or location of a polypeptide are known to one of skill in the art, e.g. Western blotting with an antibody can be used to determine the level of a polypeptide. Antibodies to CALR are commercially available, e.g., EPR3924 (Cat No. ab92516) and FMC 75 (Cat. No. ab22683) from AbCam (Cambridge, MA).

In some embodiments of any of the aspects, the at least one CALR agonist is selected from the group consisting of: DL-threo-1-Phenyl-2-palmitoylamino-3-morpholino-1-propanol; cambinol; ganciclovir; 8-bromo-GMP; arvanil; thiocitrulline; isoguvacine; ibudilast; zaprinast; FK-520; AG1478; bongkrekic acid; GW9508; fusidic acid; beta-lapachone; fusaric acid; fenspiride; zardaverine; docosahexaenoic acid, loxoprofen; AG1480; flufenamic acid; and MG-132. In some embodiments of any of the aspects, the at least one CALR agonist is selected from the group consisting of: DL-threo-1-Phenyl-2-palmitoylamino-3-morpholino-1-propanol; ganciclovir; 8-bromo-GMP; arvanil; thiocitrulline; isoguvacine; ibudilast; zaprinast; FK-520; AG1478; bongkrekic acid; GW9508; fusidic acid; beta-lapachone; fusaric acid; fenspiride; zardaverine; docosahexaenoic acid, loxoprofen; AG1480; flufenamic acid; and MG-132.

In some embodiments of any of the aspects, the at least one CALR agonist is one CALR agonist. In some embodiments of any of the aspects, the at least one CALR agonist is two CALR agonists. In some embodiments of any of the aspects, the at least one CALR agonist is three CALR agonists. In some embodiments of any of the aspects, the at least one CALR agonist is four or more CALR agonists. Any combination of two, three, four, or more CALR agonists described herein is contemplated herein

In some embodiments of any of the aspects, the CALR agonist is DL-threo-1-Phenyl-2-palmitoylamino-3-morpholino-1-propanol. In some embodiments of any of the aspects, the CALR agonist is cambinol (5-[(2-hydroxynaphthalen-1-yl)methyl]-6-phenyl-2-sulfanylidene-1H-pyrimidin-4-one). In some embodiments of any of the aspects, the CALR agonist is 2-Nor-2′-deoxyguanosine (Ganciclovir). In some embodiments of any of the aspects, the CALR agonist is 8-bromo-GMP (8-bromo-guanosine monophosphate). In some embodiments of any of the aspects, the CALR agonist is arvanil (also known in the art as N-vanillylarachidonamide and N-arachidonoyl vanillylamine). In some embodiments of any of the aspects, the CALR agonist is N⁵-[imino(methylthio)methyl]-L-ornithine, dihydrochloride (thiocitrulline). In some embodiments of any of the aspects, the CALR agonist is isoguvacine (1,2,3,6-tetrahydropyridine-4-carboxylic acid). In some embodiments of any of the aspects, the CALR agonist is ibudilast (2-methyl-1-(2-propan-2-ylpyrazolo[1,5-a]pyridin-3-yl)propan-1-one). In some embodiments of any of the aspects, the CALR agonist is 4-[[(3-phenoxyphenyl)methyl]amino]-benzenepropanoic acid (also known as (3-[4-[(3-phenoxyphenyl)methylamino]phenyl]propanoic acid) (GW9508). In some embodiments of any of the aspects, the CALR agonist is zaprinast (5-(2-propoxyphenyl)-2,6-dihydrotriazolo[4,5-d]pyrimidin-7-one). In some embodiments of any of the aspects, the CALR agonist is FK-520 (ascomycin). In some embodiments of any of the aspects, the CALR agonist is AG1478 (N-(3-chlorophenyl)-6,7-dimethoxyquinazolin-4-amine). In some embodiments of any of the aspects, the CALR agonist is bongkrekic acid ((2E,4Z,6R,8Z,10E,14E,17S,18E,20Z)-20-(carboxymethyl)-6-methoxy-2,5,17-trimethyldocosa-2,4,8,10,14,18,20-heptaenedioic acid). In some embodiments of any of the aspects, the CALR agonist is fusidic acid. In some embodiments of any of the aspects, the CALR agonist is beta-lapachone (2,2-dimethyl-3,4-dihydrobenzo[h]chromene-5,6-dione). In some embodiments of any of the aspects, the CALR agonist is fusaric acid. In some embodiments of any of the aspects, the CALR agonist is 8-(2-phenylethy)-1-oxa-3,8-diazaspiro[4.5]decan-2-one, monohydrochloride (fenspiride) In some embodiments of any of the aspects, the CALR agonist is 6-[4-(difluoromethoxy)-3-methoxyphenyl]-3(2H)-pyridazinone (zardaverine). In some embodiments of any of the aspects, the CALR agonist is docosahexaenoic acid. In some embodiments of any of the aspects, the CALR agonist is loxoprofen ((RS)-2-{4-[(2-oxocyclopentyl)methyl]phenyl}propanoic acid). In some embodiments of any of the aspects, the CALR agonist is AG1480. In some embodiments of any of the aspects, the CALR agonist is flufenamic acid (2-[3-(trifluoromethyl)anilino]benzoic acid). In some embodiments of any of the aspects, the CALR agonist is MG-132 (benzyl N-[(2S)-4-methyl-1-[[(2S)-4-methyl-1-[[(2S)-4-methyl-1-oxopentan-2-yl]aamino]-1-oxopentan-2-yl]amino]-1-oxopentan-2-yl]carbamate).

As described herein, a CALR agonist as described herein can stimulate the mitochondria, resulting in reactive oxygen species (ROS) production. In some embodiments of any of the aspects, contacting a cell or administering a subject a CALR agonist as described herein can stimulate the mitochondria, resulting in an increased level of reactive oxygen species (ROS) as compared to prior to the contacting or administration. In some embodiments of any of the aspects, contacting a cell or administering a subject a CALR agonist as described herein can stimulate the mitochondria, resulting in an increased level of reactive oxygen species (ROS) production as compared to prior to the contacting or administration.

It is contemplated herein that the mitochondrial stimulation can be modulation of mitochondrial membrane potential. It is contemplated herein that the mitochondrial stimulation can be modulation of mitochondrial ROS production. It is contemplated herein that the mitochondrial stimulation can be an increase of mitochondrial membrane potential. It is contemplated herein that the mitochondrial stimulation can be an increase of mitochondrial ROS production.

As described in the examples herein, some CALR agonists do stimulate reactive oxygen species (ROS) production; or stimulate ROS production to statistically significant levels. This group of CALR agonists are referred to herein as “ROS+ CALR agonists.” ROS+ CALR agonists include Ibudilast; zaprinast; FK-520; AG1478; bongkrekic acid; GW9508; fusidic acid; beta-lapachone; fusaric acid; fenspiride; zardaverine; docosahexaenoic acid, loxoprofen; AG1480; flufenamic acid; and MG-132.

In some embodiments of any of the aspects, the at least one CALR agonist comprises at least one ROS+ CALR agonist. In some embodiments of any of the aspects, the at least one CALR agonist is a ROS+ CALR agonist. In some embodiments of any of the aspects, the at least one CALR agonist does not comprise a ROS+ CALR agonist.

As described in the examples herein, some CALR agonists do not stimulate reactive oxygen species (ROS) production; or stimulate ROS production only to negligible levels. This group of CALR agonists are referred to herein as “ROS− CALR agonists.” ROS-CALR agonists include DL-threo-1-Phenyl-2-palmitoylamino-3-morpholino-1-propanol; cambinol; ganciclovir; 8-bromo-GMP; arvanil; thiocitrulline; and isoguvacine. In some embodiments, an at least one ROS− CALR agonist can be selected from the group consisting of: DL-threo-1-Phenyl-2-palmitoylamino-3-morpholino-1-propanol; ganciclovir; 8-bromo-GMP; arvanil; thiocitrulline; and isoguvacine.

In some embodiments of any of the aspects, the at least one CALR agonist comprises at least one ROS− CALR agonist. In some embodiments of any of the aspects, the at least one CALR agonist is a ROS− CALR agonist. In some embodiments of any of the aspects, the at least one CALR agonist does not comprise a ROS− CALR agonist.

As described herein, CALR agonists can increase the interaction of HSCs with macrophages, promoting the proliferation of healthy HSCs and reducing the proliferation of pathological or unhealthy HSCs. Accordingly, in one aspect of any of the embodiments, described herein is a method of improving or increased proliferation and/or survival of healthy hematopoietic stem cells, the method comprising contacting a population of hematopoietic stem cells (HSCs) with a CALR agonist, e.g., a ROS− CALR agonist.

As used herein, “proliferating” and “proliferation” refers to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

As used herein, “survival” refers to the ability of a cell or population to maintain viability over time. Survival can encompass proliferation and/or a failure to experience cell death.

As used herein, the term “hematopoietic stem cells” (or “HSCs”) refer to immature blood cells having the capacity to self-renew and to differentiate into mature blood cells comprising diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34⁺ cells. CD34⁺ cells are immature cells that express the CD34 cell surface marker. In humans, CD34⁺ cells are believed to include a subpopulation of cells with the stem cell properties defined above, whereas in mice, HSCs are CD34−.

Hematopoietic stem cells are optionally obtained from blood products. A blood product includes a product obtained from the body or an organ of the body containing cells of hematopoietic origin. Such sources include unfractionated bone marrow, umbilical cord, placenta, peripheral blood, or mobilized-peripheral blood. All of the aforementioned crude or unfractionated blood products can be enriched for cells having hematopoietic stem cell characteristics in a number of ways. For example, the more mature, differentiated cells are selected against, via cell surface molecules they express. Optionally, the blood product is fractionated by positively selecting for CD34⁺ cells. CD34⁺ cells include a subpopulation of hematopoietic stem cells capable of self-renewal, multi-potency, and that can be reintroduced into a transplant recipient whereupon they home to the hematopoietic stem cell niche and re-establish productive and sustained hematopoiesis. Such selection is accomplished using, for example, commercially available magnetic anti-CD34 beads (Dynal, Lake Success, N. Y.). Unfractionated blood products are optionally obtained directly from a donor or retrieved from cryopreservative storage. Hematopoietic stem cells can also be optionally obtained from differentiated embryonic stem cells, differentiated induced pluripotent stem cells or from other reprogrammed mature cell types.

A “healthy HSC” refers to an HSC exhibiting a rate of proliferation and progeny differentiation within the average or non-pathological rates for HSCs under similar conditions. This is contrasted with unhealthy HSCs, e.g., those exhibiting abnormally high proliferation rates which contributed to clonal hematopoiesis pathologies.

In embodiments related to contacting a population of hematopoietic stem cells (HSCs), the contacting can occur ex vivo. The HSCs can be isolated HSCs, cultured HSCs, an HSCs cell line, HSCs differentiated from a more pluripotent stem cell (e.g., embryonic stem cell, induced pluripotent stem cell, or mesodermal stem cell), or provided in a sample obtained from a subject (e.g., a bone marrow sample comprising one or more cell types). In some embodiments of any of the aspects, the contacting can comprise maintaining a concentration or presence of the at least one CALR agonist in a culture or medium comprising the population of HSCs. In some embodiments of any of the aspects, the contacting can comprise intermittently adding the at least one CALR agonist to a culture or medium comprising the population of HSCs, e.g., by injection, aliquoting, or fluidics.

In some embodiments, the at least one CALR agonist is present in an amount sufficient to increase proliferation of the population of cells by 10% or more relative to a population of hematopoietic stem cells not contacted with the at least one CALR agonist after seven or more days of culture (e.g., after seven, ten, twelve, fourteen, fifteen, twenty, or more days of culture). In some embodiments, the hematopoietic stem cells are cultured for two or more days (e.g., three, five, seven, ten, twelve, fourteen, fifteen, twenty or more days). In some embodiments, the hematopoietic stem cells contact the at least one CALR agonist for two or more days (e.g., three, five, seven, ten, twelve, fourteen, fifteen, twenty, or more days).

In some embodiments, the hematopoietic stem cells are mammalian cells, such as human cells. In some embodiments, the hematopoietic stem cells are CD34+ cells. In some embodiments, the hematopoietic stem cells are from human cord blood. In some embodiments, the hematopoietic stem cells are from human mobilized peripheral blood. In some embodiments, the hematopoietic stem cells are from human bone marrow. In some embodiments, the hematopoietic stem cells are freshly isolated from a human. In some embodiments, the hematopoietic stem cells have been previously cryopreserved.

In some embodiments, the population of HSCs and/or the progeny of the population of HSCs is subsequently administered to a subject. The HSCs can be autologous to the subject. The HSCs can be originally obtained from a source other than the subject, e.g., a donor.

Alternatively, or in addition, the contacting can comprise administering the CALR agonist to a subject in need of improved proliferation and/or survival of healthy hematopoietic stem cells. In some embodiments of any of the aspects, the subject in need of improved proliferation and/or survival of healthy hematopoietic stem cells is a subject in need of or receiving a transplant or adoptive cell therapy.

In some embodiments of the methods of the invention, the subject in need of improved proliferation and/or survival of healthy hematopoietic stem cells is a human patient suffering from a disease selected from the group consisting of Acute Lymphoblastic Leukemia (ALL), Acute Myelogenous Leukemia (AML), Chronic Myelogenous Leukemia (CML), Chronic Lymphocytic Leukemia (CLL), Hodgkin Lymphoma (HL), Non-Hodgkin Lymphoma (NHL), Myelodysplastic Syndrome (MDS), Multiple myeloma, Aplastic anemia, Bone marrow failure, Myeloproliferative disorders such as Myelofibrosis, Essential thrombocytopenia or Polycythemia vera, Fanconi anemia, Dyskeratosis congenita, Common variable immune deficiency (CVID, such as CVID 1, CVID 2, CVID 3, CVID 4, CVID 5, and CVID 6), Human immunodeficiency virus (HIV), Hemophagocytic lymphohistiocytosis, Amyloidosis, Solid tumors such as Neuroblastoma, Germ cell tumors, Breast cancer, Wilms' tumor, Medulloblastoma, and Neuroectodermal tumors, Autoimmune diseases such as Scleroderma, Multiple sclerosis, Ulcerative colitis, Systemic lupus erythematosus and Type I diabetes, or protein deficiencies such as Adrenoleukodystrophy (ALD), Metachromatic leukodystrophy (MLD), Hemophilia A & B, Hurler syndrome, Hunter syndrome, Fabry disease, Gaucher disease, Epidermolysis bullosa, Globoid Cell Leukodystrophy, Sanfillipo syndrome, and Morquio syndrome.

In some embodiments of the methods of the invention, the subject in need of improved proliferation and/or survival of healthy hematopoietic stem cells is a human patient suffering from a genetic blood disease selected from the group consisting of Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease (X-linked Chronic granulomatous disease, autosomal recessive (AR) chronic granulomatous disease, chronic granulomatous disease AR I NCF1, Chronic granulomatous disease AR CYBA, Chronic granulomatous disease AR II NCF2, Chronic granulomatous disease AR III NCF4), X-linked Severe Combined Immune Deficiency (SCID), ADA SCID, IL7-RA SCID, CD3 SCID, Rag1/Rag2 SCID, Artemis SCID, CD45 SCID, Jak3 SCID, Congenital agranulocytosis, Congenital agranulocytosis-congenital neutropenia—SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Unicentric Castleman's Disease, Multicentric Castleman's Disease, Congenital amegakaryocytic thrombocytopenia (CAMT) type I, Reticular dysgenesis, Fanconi anemia, Acquired idiopathic sideroblastic anemia, Systemic mastocytosis, Von willebrand disease (VWD), Congenital dyserythropoietic anemia type 2, Cartilage-hair hypoplasia syndrome, Hereditary spherocytosis, Blackfan-Diamond syndrome, Shwachman-Diamond syndrome, Thrombocytopenia-absent radius syndrome, Osteopetrosis, Infantile osteopetrosis, Mucopolysaccharidoses, Lesch-Nyhan syndrome, Glycogen storage disease, Congenital mastocytosis, Omenn syndrome, X-linked Immunodysregulation, polyendocrinopathy, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, and Bare lymphocyte Syndrome type II (Bare lymphocyte Syndrome type II, MHC class I deficiency; Bare lymphocyte Syndrome type II, complementation group A; Bare lymphocyte Syndrome type II, complementation group C; Bare lymphocyte Syndrome type II complementation group D; Bare lymphocyte Syndrome type II, complementation group E).

In some embodiments of the methods of the invention, the subject in need of improved proliferation and/or survival of healthy hematopoietic stem cells is a human patient suffering from a hematolymphoid malignancy, a non-hematolymphoid malignancy, or a protein deficiency, or a tissue or cell transplantation recipient (e.g., to induce tolerance to transplanted tissue or cells).

Populations of hematopoietic stem cells contacted with at least one CALR agonist by the methods of the invention, as well as progeny thereof, can also be used to treat a patient (e.g., a human patient) suffering from a hematolymphoid malignancy, a non-hematolymphoid malignancy, or a protein deficiency. In other embodiments, the patient may be the recipient of a tissue or cell transplant, and the hematopoietic stem cells or progeny thereof are administered in order to induce tolerance to the transplanted tissue or cells.

In some embodiments of the above-described methods of treating a patient with hematopoietic stem cells or progeny thereof, the hematopoietic stem cells are autologous or syngeneic. Alternatively, the hematopoietic stem cells may be allogeneic.

In some embodiments, the methods described herein relate to treating a subject having or diagnosed as having a need for reduced hematopoiesis with at least one CALR agonist. Subjects having a need for reduced hematopoiesis can be identified by a physician using current methods of diagnosing aberrant hematopoiesis. Symptoms and/or complications of aberrant hematopoiesis which characterize these conditions and aid in diagnosis are well known in the art for the conditions described herein. Tests that may aid in a diagnosis of a condition described herein include, but are not limited to, blood counts or clonal analysis. A family history of a need for reduced hematopoiesis, or exposure to risk factors for aberrant hematopoiesis can also aid in determining if a subject is likely to have a need for reduced hematopoiesis or in making a diagnosis of aberrant hematopoiesis (e.g., one or more of the conditions described herein).

The compositions and methods described herein can be administered to a subject having or diagnosed as having a need for reduced hematopoiesis. In some embodiments, the methods described herein comprise administering an effective amount of compositions described herein, e.g. at least one CALR agonist to a subject in order to alleviate a symptom of a disease described herein. As used herein, “alleviating a symptom” is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

In some embodiments of any of the aspects, the administration is oral. In some embodiments of any of the aspects, the administration is intravenous.

The term “effective amount” as used herein refers to the amount of at least one CALR agonist needed to alleviate at least one or more symptom of the disease or disorder, and relates to a sufficient amount of pharmacological composition to provide the desired effect. The term “therapeutically effective amount” therefore refers to an amount of at least one CALR agonist that is sufficient to provide a particular anti-hematopoiesis effect when administered to atypical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient(s), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., blood counts, and/or an assay for hematopoiesis and hematopoietic clonality, among others. The dosage can be determined by a physician and adjusted, as necessary, to suit observed effects of the treatment.

In some embodiments, the technology described herein relates to a pharmaceutical composition comprising at least one CALR agonist as described herein, and optionally a pharmaceutically acceptable carrier. In some embodiments, the active ingredients of the pharmaceutical composition comprise at least one CALR agonist as described herein. In some embodiments, the active ingredients of the pharmaceutical composition consist essentially of at least one CALR agonist as described herein. In some embodiments, the active ingredients of the pharmaceutical composition consist of at least one CALR agonist as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. at least one CALR agonist as described herein.

In some embodiments, the pharmaceutical composition comprising at least one CALR agonist as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of at least one CALR agonist as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of at least one CALR agonist as disclosed herein can also be incorporated into the parenteral dosage forms of the disclosure, including conventional and controlled-release parenteral dosage forms.

Pharmaceutical compositions comprising at least one CALR agonist can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the at least one CALR agonist can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif USA)), or a combination thereof to provide the desired release profile in varying proportions.

In some embodiments of any of the aspects, the at least one CALR agonist described herein is administered as a monotherapy, e.g., another treatment for a condition described herein is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In addition, the methods of treatment can further include the use of radiation or radiation therapy. Further, the methods of treatment can further include the use of surgical treatments.

In some embodiments of any of the aspects, the method further comprises administering to the subject at least one chemotherapeutic. In some embodiments of any of the aspects, the at least one chemotherapeutic is selected from 5-azacytidine; venetoclax; guadecitabine

In certain embodiments, an effective dose of a composition comprising at least one CALR agonist as described herein can be administered to a patient once. In certain embodiments, an effective dose of a composition comprising at least one CALR agonist can be administered to a patient repeatedly. For systemic administration, subjects can be administered a therapeutic amount of a composition comprising at least one CALR agonist, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. hematopoiesis by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient(s). The desired dose or amount of activation can be administered at one time or divided into sub doses, e.g., 2-4 sub doses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising at least one CALR agonist can be administered over a period of time, such as over a 5 minute, 10 minute, 15 minute, 20 minute, or 25 minute period.

The dosage ranges for the administration of at least one CALR agonist, according to the methods described herein depend upon, for example, the form of the at least one CALR agonist, its potency, and the extent to which symptoms, markers, or indicators of a condition described herein are desired to be reduced, for example the percentage reduction desired for hematopoiesis or the extent to which, for example, HSC quality assurance macrophage activity is desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of at least one CALR agonist in, e.g. the treatment of a condition described herein, or to induce a response as described herein (e.g. reduced hematopoiesis) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. blood counts. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. rate or clonality of hematopoiesis). It is well within the ability of one skilled in the art to monitor efficacy of administration and/or treatment by measuring any one of such parameters, or any combination of parameters. Efficacy can be assessed in animal models of a condition described herein, for example treatment of any of the conditions described herein. When using an experimental animal model, efficacy of treatment is evidenced when a statistically significant change in a marker is observed, e.g. the rate or clonality of hematopoiesis.

In vitro and animal model assays are known which allow the assessment of a given dose of at least one CALR agonist. By way of non-limiting example, the effects of a dose of at least one CALR agonist can be assessed by administering it to a mouse or primate model of any of the conditions described herein and measuring the subsequent rate and/or clonality of hematopoiesis.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element did not deem an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually, the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits, and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish, and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of the conditions described herein. A subject can be male or female. In some embodiments, the subject is a human.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition, or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification, and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The terms “stem cell” or “undifferentiated cell” as used herein, refer to a cell in an undifferentiated or partially differentiated state that has the property of self-renewal and has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retro differentiation” by persons of ordinary skill in the art.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. one of the conditions described herein. The term “treating” includes reducing or alleviating at least one adverse effect or symptom of a condition, disease or disorder described herein. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, treatment is effective if the rate of hematopoiesis in a subject is decreased by a statistically significant amount after administration of at least one CALR agonist described herein. In some embodiments of any of the aspects, treatment is effective if the degree of clonality of hematopoiesis in a subject is decreased by a statistically significant amount after administration of at least one CALR agonist described herein.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two-standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In all embodiments where a sample is obtained or has been obtained or provided, the sample can be sample taken, obtained, or provided via minimally invasive methods and/or involves only a minor intervention. In some embodiments of any of the aspects, a sample is taken, obtained, or provided by one or more of a blood draw or prick, an epidermal or mucus membrane swab, buccal sampling, saliva sample, a epidermal skin sampling technique, and/or collection of a secreted or expelled bodily fluid (e.g., mucus, urine, sweat, etc), fecal sampling, semen/seminal fluid sampling, or clippings (e.g., of hair or nails). In some embodiments of any of the aspects, the sample comprises, consists of, or consists essentially of blood (or any fraction or component thereof), serum, urine, mucus, epithelial cells, saliva, buccal cells, a secreted or expelled bodily fluid, and/or hair or nail clippings.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A method of reducing hematopoiesis in a subject in need thereof, the method comprising administering at least one CALR agonist to the subject.
2. The method of paragraph 1, wherein the subject is a subject in need of treatment for clonal hematopoiesis, Clonal Hematopoiesis of Indeterminate Potential (CHIP), myelodysplastic syndrome (MDS), or leukemia.
3. The method of any one of the preceding paragraphs, wherein the hematopoiesis is pathological hematopoiesis.
4. The method of any one of the preceding paragraphs, wherein the administration is oral.
5. The method of any one of the preceding paragraphs, wherein the administration is intravenous.
6. The method of any one of the preceding paragraphs, whereby mitochondrial stimulation produces ROS.
7. The method of paragraph 6, wherein mitochondrial stimulation comprises mitochondrial modulation.
8. The method of any one of the preceding paragraphs, wherein the at least one CALR agonist is at least one ROS− CALR agonist.
9. The method of paragraph 8, wherein the at least one ROS− CALR agonist is selected from the group consisting of:
   DL-threo-1-Phenyl-2-palmitoylamino-3-morpholino-1-propanol; cambinol; ganciclovir; 8-bromo-GMP; arvanil; thiocitrulline; and isoguvacine.
10. The method of any one of the preceding paragraphs, wherein the at least one CALR agonist is at least one ROS+ CALR agonist.
11. The method of paragraph 10, wherein the at least one ROS+ CALR agonist is selected from the group consisting of:
   Ibudilast; zaprinast; FK-520; AG1478; bongkrekic acid; GW9508; fusidic acid; beta-lapachone; fusaric acid; fenspiride; zardaverine; docosahexaenoic acid, loxoprofen; AG1480; flufenamic acid; and MG-132.
12. The method of any one of the preceding paragraphs, the method further comprising administering to the subject at least one chemotherapeutic.
13. The method of paragraph 12, wherein the at least one chemotherapeutic is selected from 5-azacytidine; venetoclax; and guadecitabine.
14. A method of improving proliferation and/or survival of healthy hematopoietic stem cells, the method comprising contacting a population of hematopoietic stem cells (HSCs) with at least one CALR agonist.
15. The method of paragraph 14, wherein the at least one CALR agonist is at least one ROS− CALR agonist.
16. The method of paragraph 15, wherein the at least one ROS− CALR agonist is selected from the group consisting of:
   DL-threo-1-Phenyl-2-palmitoylamino-3-morpholino-1-propanol; cambinol; ganciclovir; 8-bromo-GMP; arvanil; thiocitrulline; and isoguvacine
17. The method of any one of paragraphs 14-16, wherein the contacting occurs ex vivo.
18. The method of paragraph 17, wherein the population of HSCs and/or the progeny of the population of HSCs is subsequently administered to a subject.
19. The method of any one of paragraphs 14-16, wherein the contacting comprises administering the at least one CALR agonist to a subject in need of improved proliferation and/or survival of healthy hematopoietic stem cells.
20. The method of paragraph 19, wherein the subject in need of improved proliferation and/or survival of healthy hematopoietic stem cells is a subject in need of or receiving a transplant or adoptive cell therapy.
21. The method of any one of paragraphs 19-20, wherein the administration is oral.
22. The method of any one of paragraphs 18-20, wherein the administration is intravenous.
23. At least one CALR agonist for use in a method of reducing hematopoiesis in a subject in need thereof.
24. The agonist of paragraph 23, wherein the subject is a subject in need of treatment for clonal hematopoiesis, Clonal Hematopoiesis of Indeterminate Potential (CHIP), myelodysplastic syndrome (MDS), or leukemia.
25. The agonist of any one of the preceding paragraphs, wherein the hematopoiesis is pathological hematopoiesis.
26. The agonist of any one of the preceding paragraphs, wherein the agonist is orally administered.
27. The agonist of any one of the preceding paragraphs, wherein the agonist is intravenously administered.
28. The agonist of any one of the preceding paragraphs, whereby mitochondrial stimulation produces ROS.
29. The agonist of paragraph 28, wherein mitochondrial stimulation comprises mitochondrial modulation.
30. The agonist of any one of the preceding paragraphs, wherein the at least one CALR agonist is at least one ROS− CALR agonist.
31. The agonist of paragraph 30, wherein the at least one ROS− CALR agonist is selected from the group consisting of:
   DL-threo-1-Phenyl-2-palmitoylamino-3-morpholino-1-propanol; cambinol; ganciclovir; 8-bromo-GMP; arvanil; thiocitrulline; and isoguvacine.
32. The agonist of any one of the preceding paragraphs, wherein the at least one CALR agonist is at least one ROS+ CALR agonist.
33. The agonist of paragraph 32, wherein the at least one ROS+ CALR agonist is selected from the group consisting of:
   Ibudilast; zaprinast; FK-520; AG1478; bongkrekic acid; GW9508; fusidic acid; beta-lapachone; fusaric acid; fenspiride; zardaverine; docosahexaenoic acid, loxoprofen; AG1480; flufenamic acid; and MG-132.
34. The agonist of any one of the preceding paragraphs, wherein the subject is further administered at least one chemotherapeutic.
35. The agonist of paragraph 34, wherein the at least one chemotherapeutic is selected from 5-azacytidine; venetoclax; and guadecitabine.
36. At least one CALR agonist for use in a method of improving proliferation and/or survival of healthy hematopoietic stem cells, the method comprising contacting a population of hematopoietic stem cells (HSCs) with at least one CALR agonist.
37. The agonist of paragraph 36, wherein the at least one CALR agonist is at least one ROS− CALR agonist.
38. The agonist of paragraph 37, wherein the at least one ROS− CALR agonist is selected from the group consisting of:
   DL-threo-1-Phenyl-2-palmitoylamino-3-morpholino-1-propanol; cambinol; ganciclovir; 8-bromo-GMP; arvanil; thiocitrulline; and isoguvacine
39. The agonist of any one of paragraphs 36-38, wherein the contacting occurs ex vivo.
40. The agonist of paragraph 39, wherein the population of HSCs and/or the progeny of the population of HSCs is subsequently administered to a subject.
41. The agonist of any one of paragraphs 36-38, wherein the contacting comprises administering the at least one CALR agonist to a subject in need of improved proliferation and/or survival of healthy hematopoietic stem cells.
42. The agonist of paragraph 41, wherein the subject in need of improved proliferation and/or survival of healthy hematopoietic stem cells is a subject in need of or receiving a transplant or adoptive cell therapy.
43. The agonist of any one of paragraphs 41-42, wherein the agonist is orally administered.
44. The agonist of any one of paragraphs 41-42, wherein the agonist is intravenously administered.

The technology described herein is further illustrated by the following examples which in no way should be construed as being further limiting.

### EXAMPLES

### Example 1

### Introduction

In many adult tissues, the maintenance of organ function depends on stem cells. Although there is a requirement of these for tissue repair, it is unknown which signals are quality assured during development. Our lab has previously identified macrophage-HSPC interactions during embryogenesis which quality assure the nascent stem cell pool. Macrophages either fully engulf stem cells or partially eat them. In the latter situation, the stem cell goes on divide. This is mediated by the “eat me” signal calreticulin (CALR) on the HSPC surface. The signals triggering CALR to the HSPC surface, mediating their removal or amplification, remain unknown.

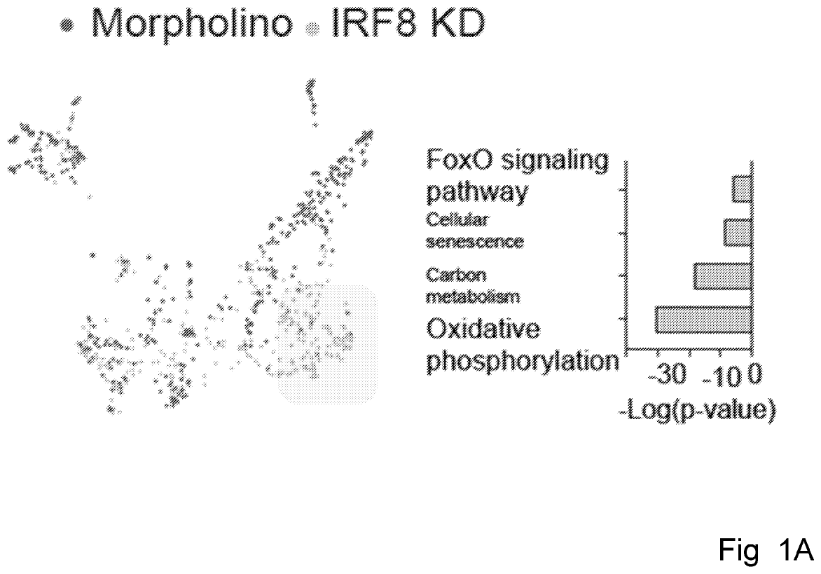

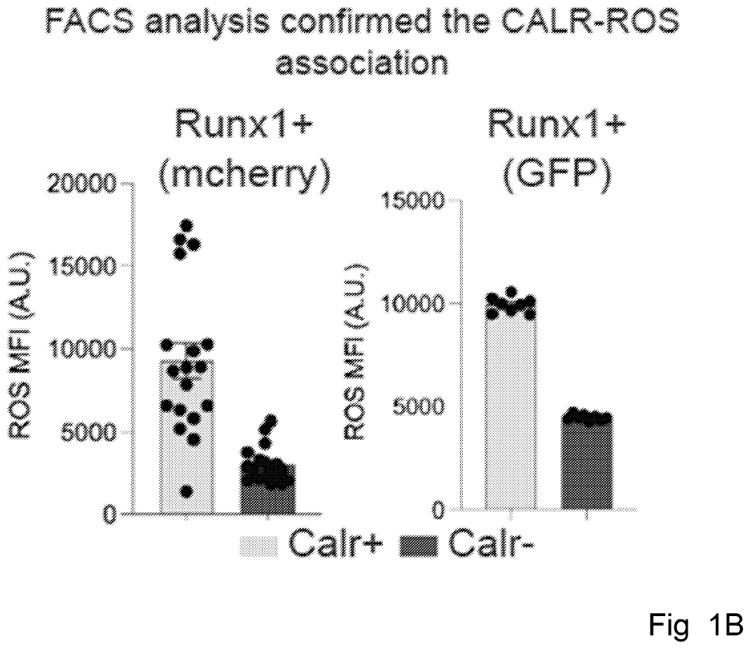

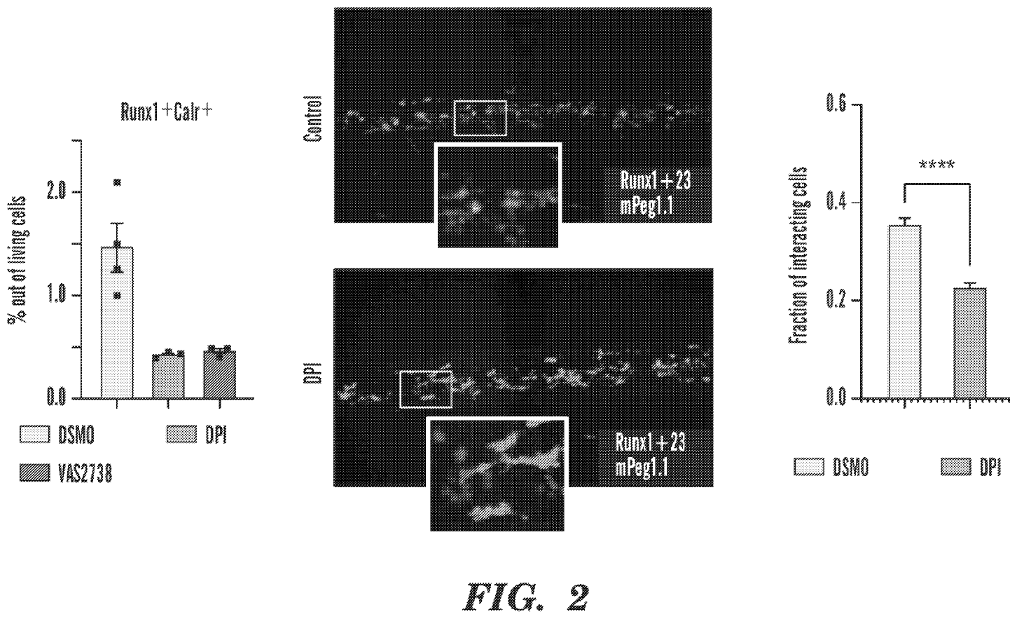

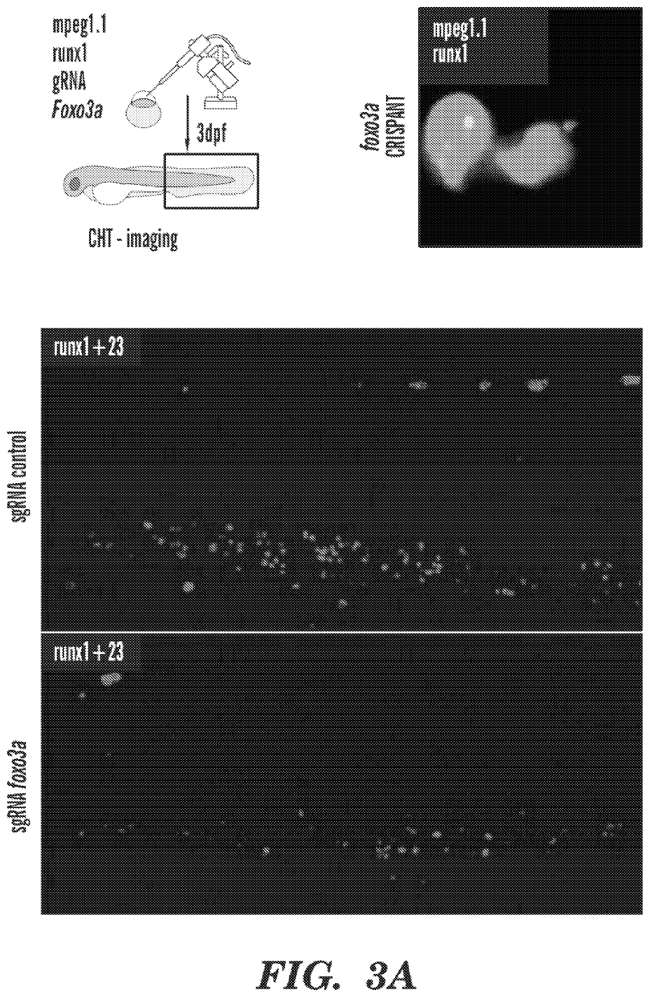

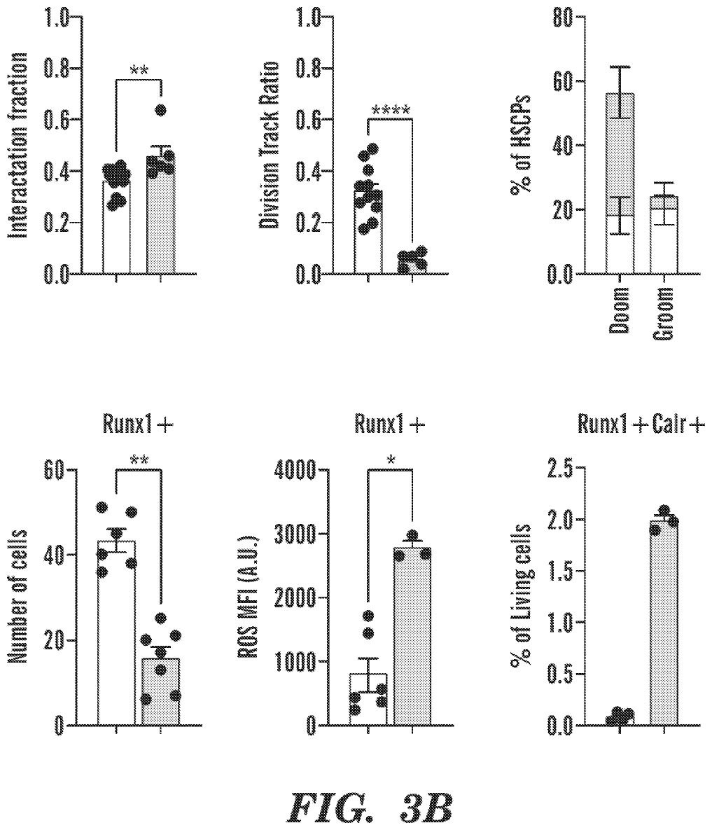

Single-cell RNAseq analysis of IRF8 KD zebrafish embryos revealed increased FoxO signaling and reactive oxygen species (FIGS. 1A-1B). Lowering ROS levels led to decrease surface CALR and reduced the stem cell-macrophage interactions (FIG. 2). Foxo3a perturbation caused increased ROS, promoted surface CALR and “dooming” events (FIGS. 3A-3B).

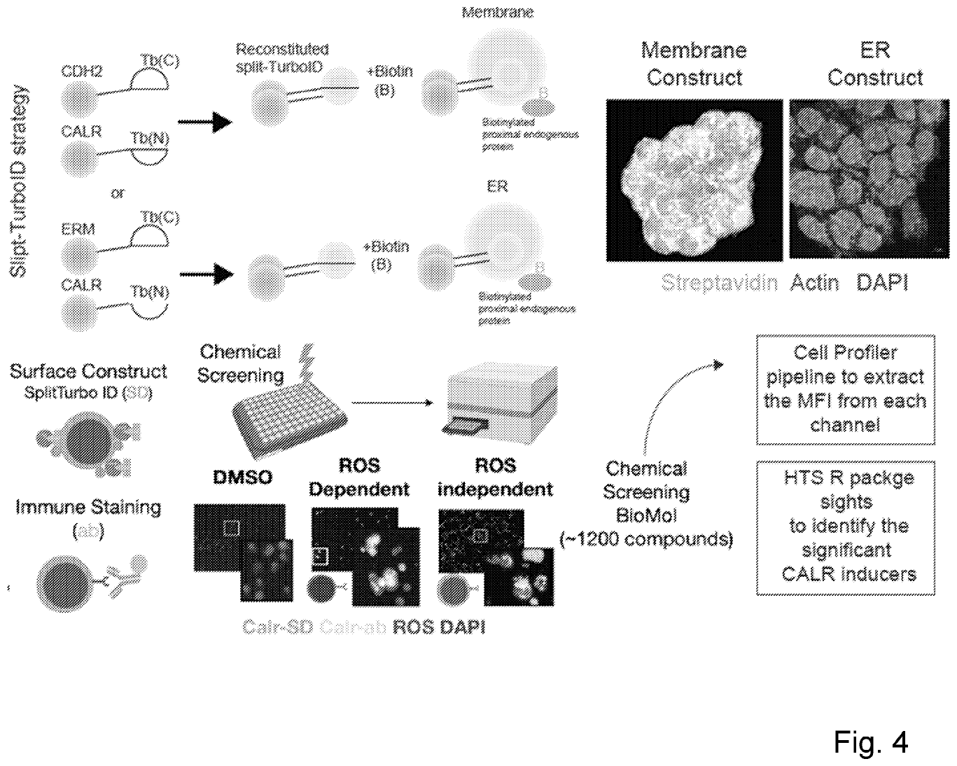

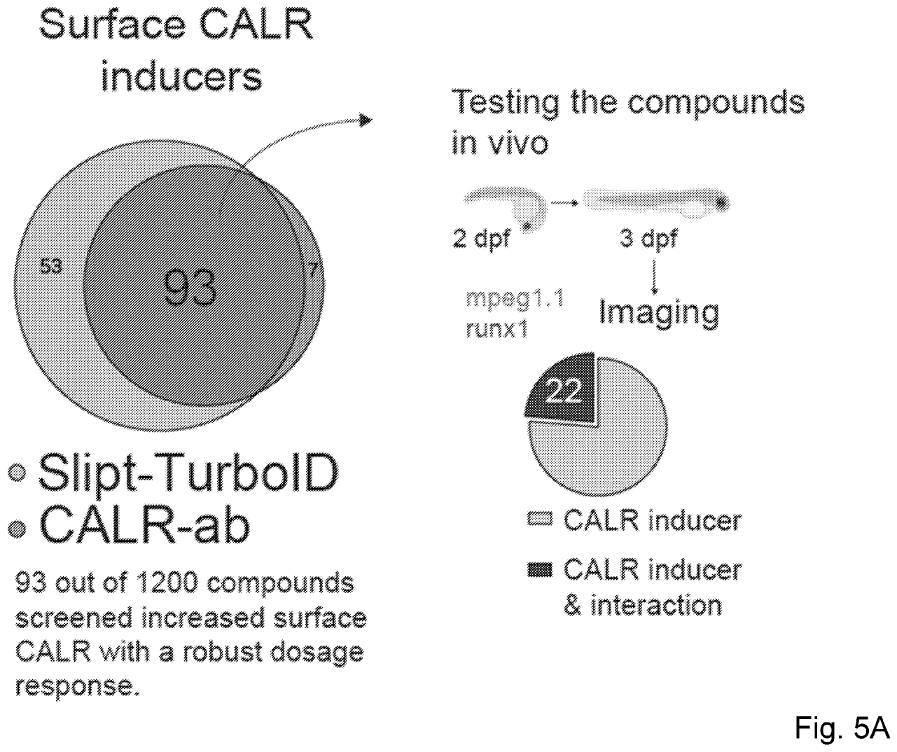

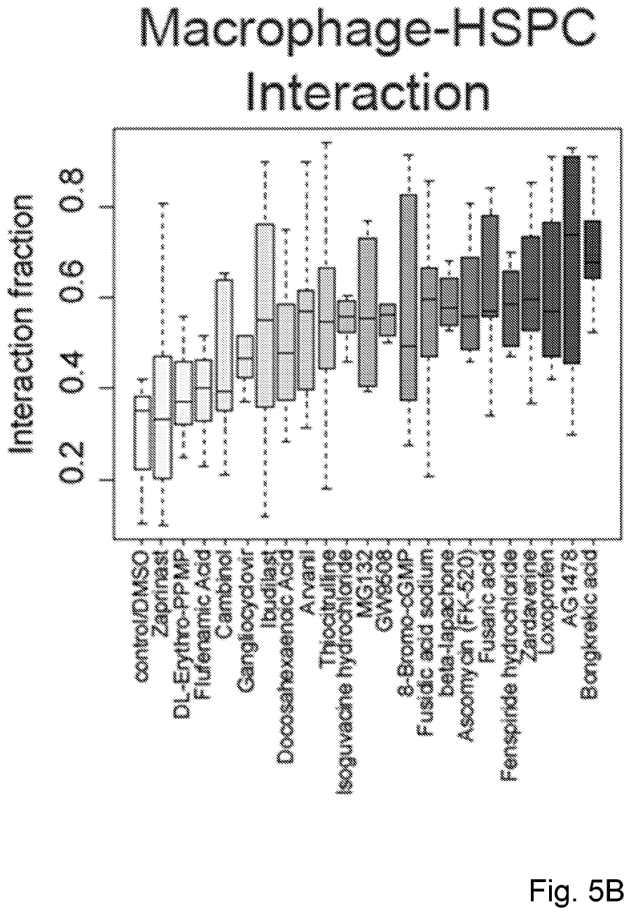

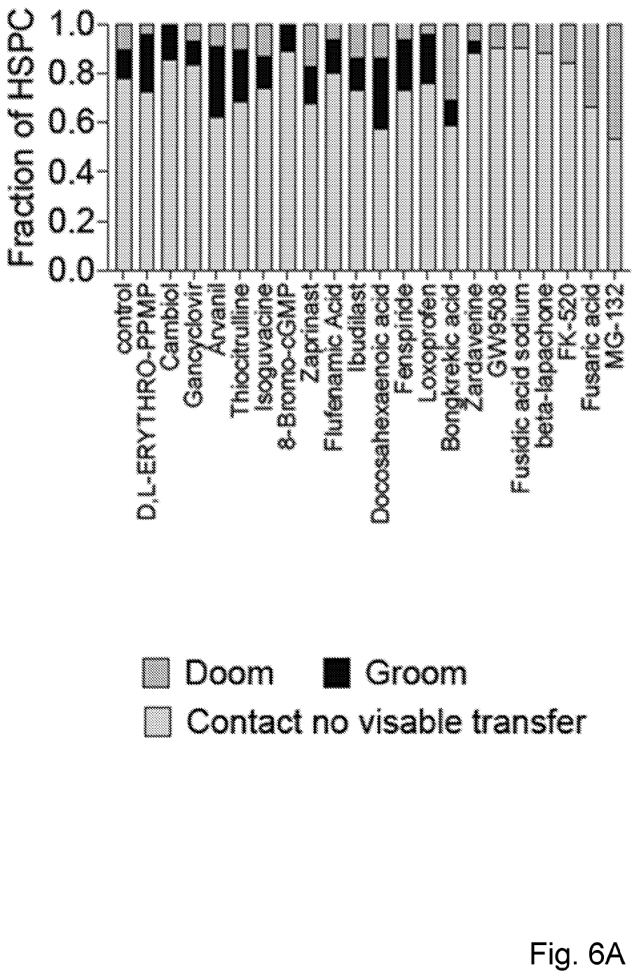

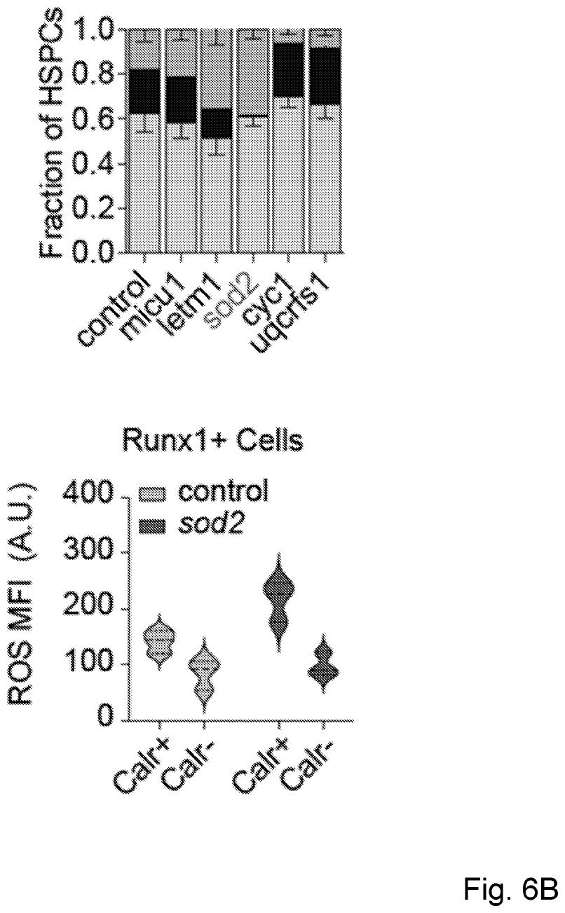

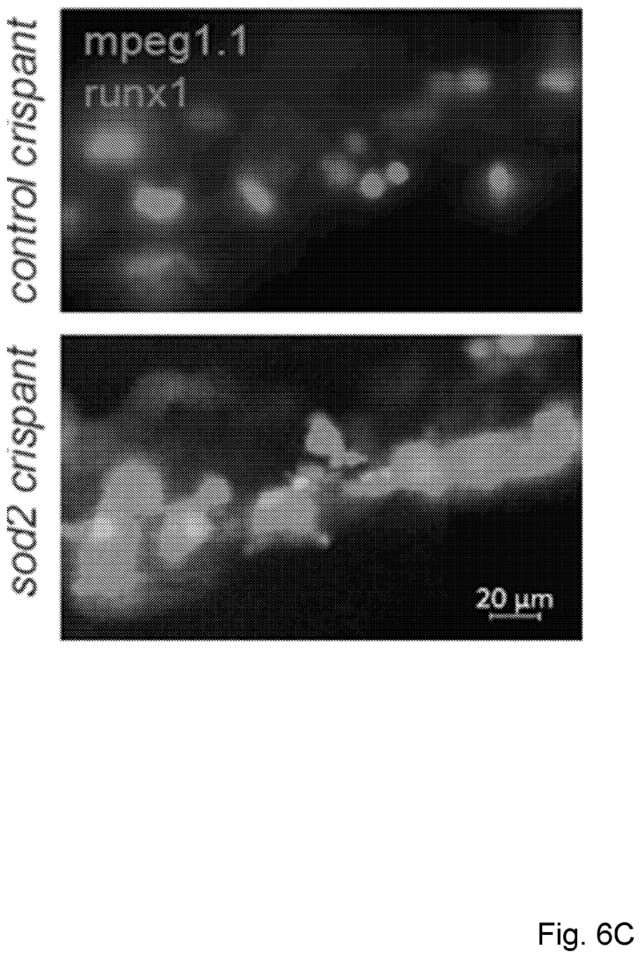

A chemical screen was conducted to identify surface CALR inducers in a ROS-associated and a ROS-independent manner (FIG. 4). Hits from the screen was validated in vivo as CALR inducers (FIGS. 5A-5B). ROS− drugs tended to induce macrophage-stem cell interaction and proliferation. ROS+ drugs tended to promote dooming (FIGS. 6A-6C).

This work demonstrates the identification of ROS as a signal that upregulated surface CALR and promoter macrophage-stem cell interactions, safeguarding the development of stem cells that are stressed or damaged.

### Example 2

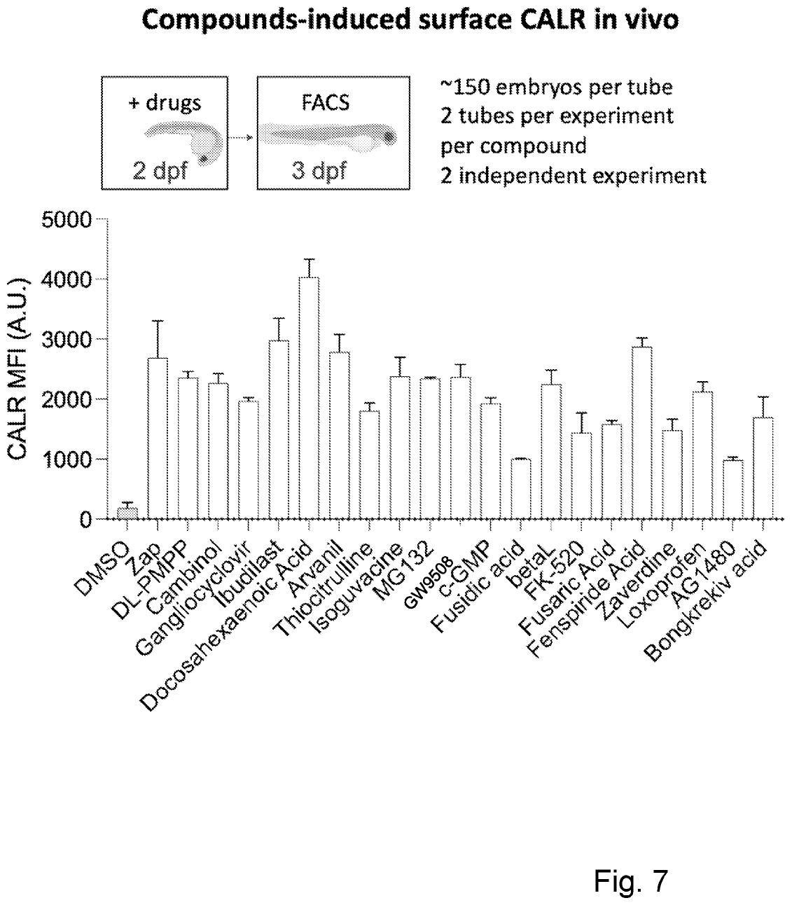

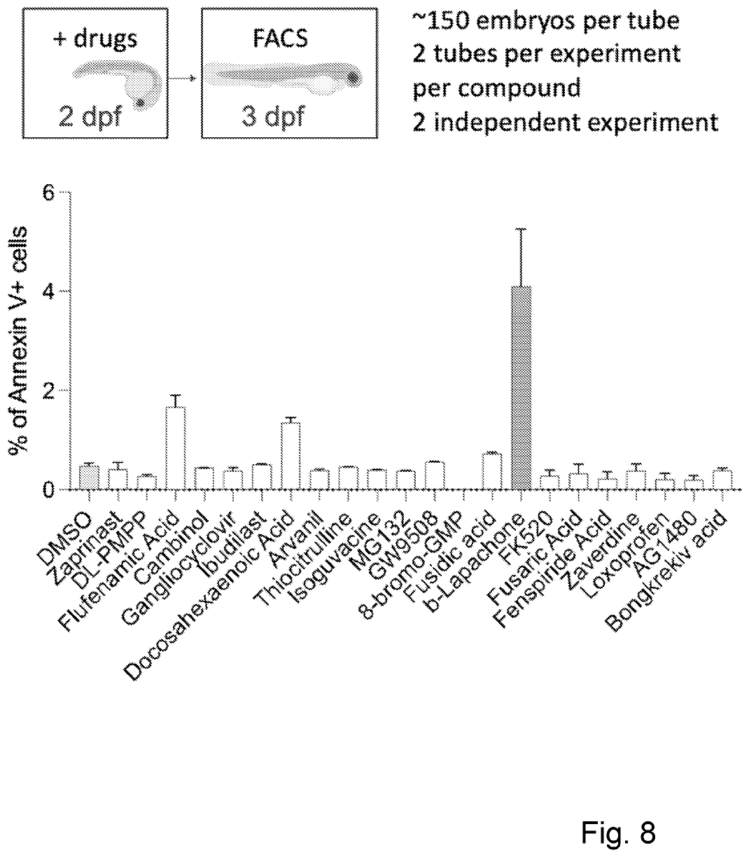

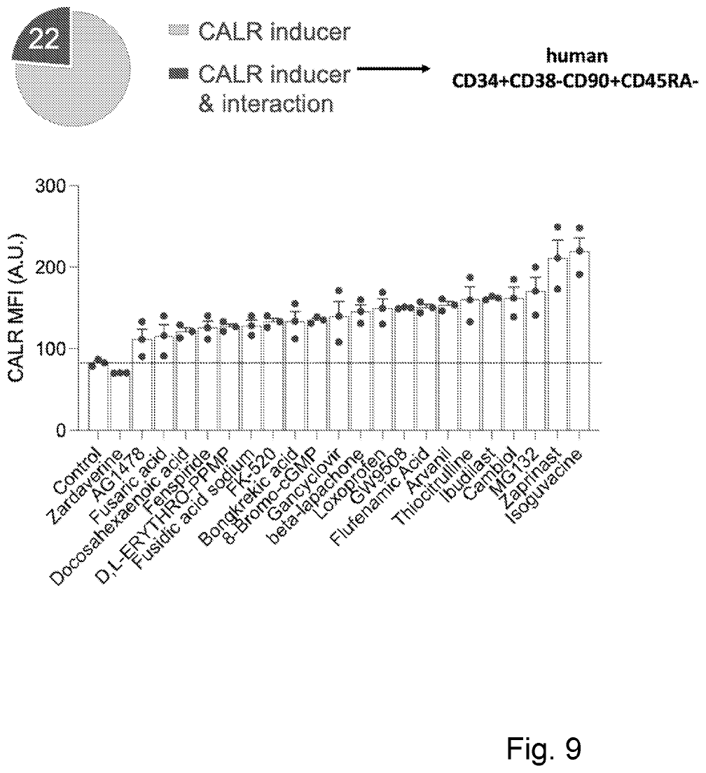

The ability of compounds to induce surface CALR in vivo was measured (FIG. 7). Only one of these compounds induced early apoptosis (FIG. 8). The compounds also induced surface CALR on human HSCs (FIG. 9).

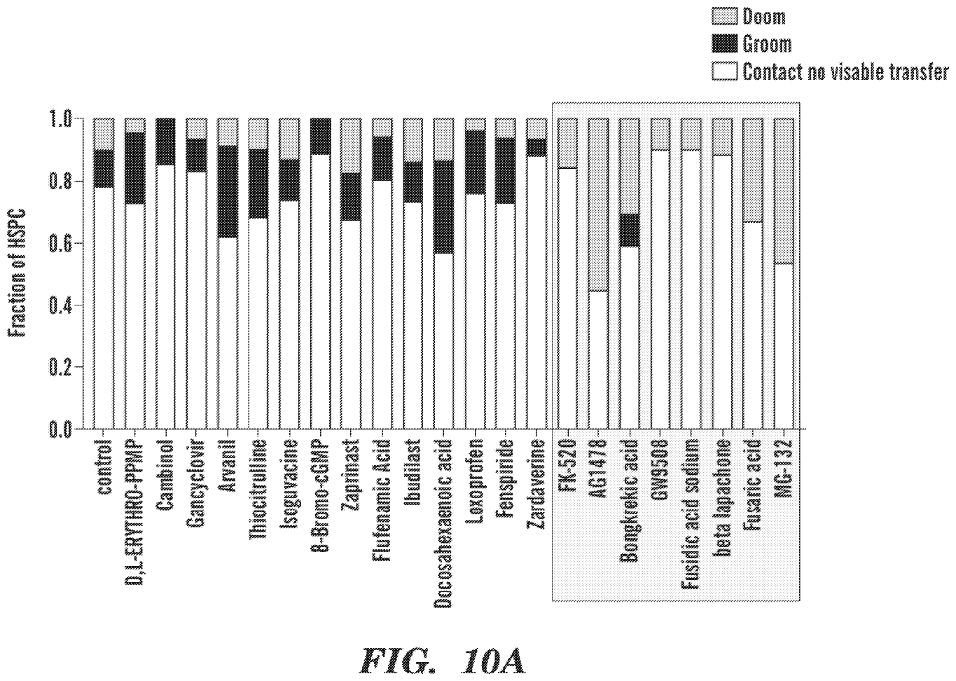

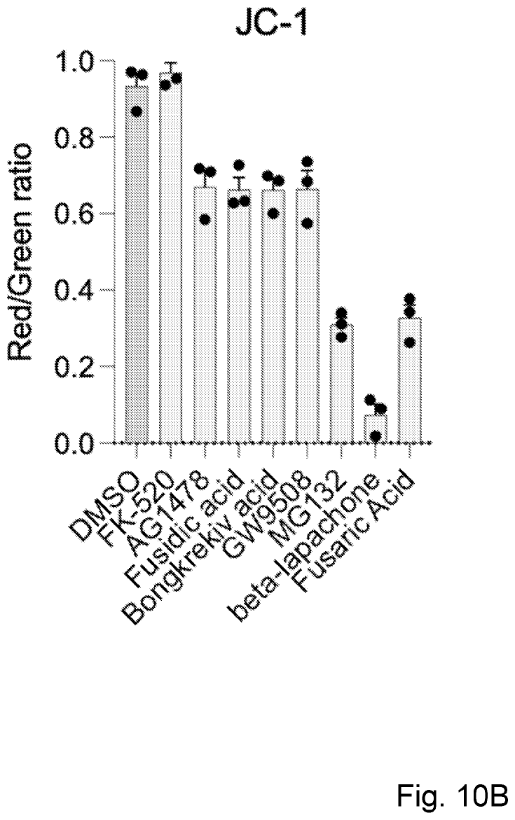

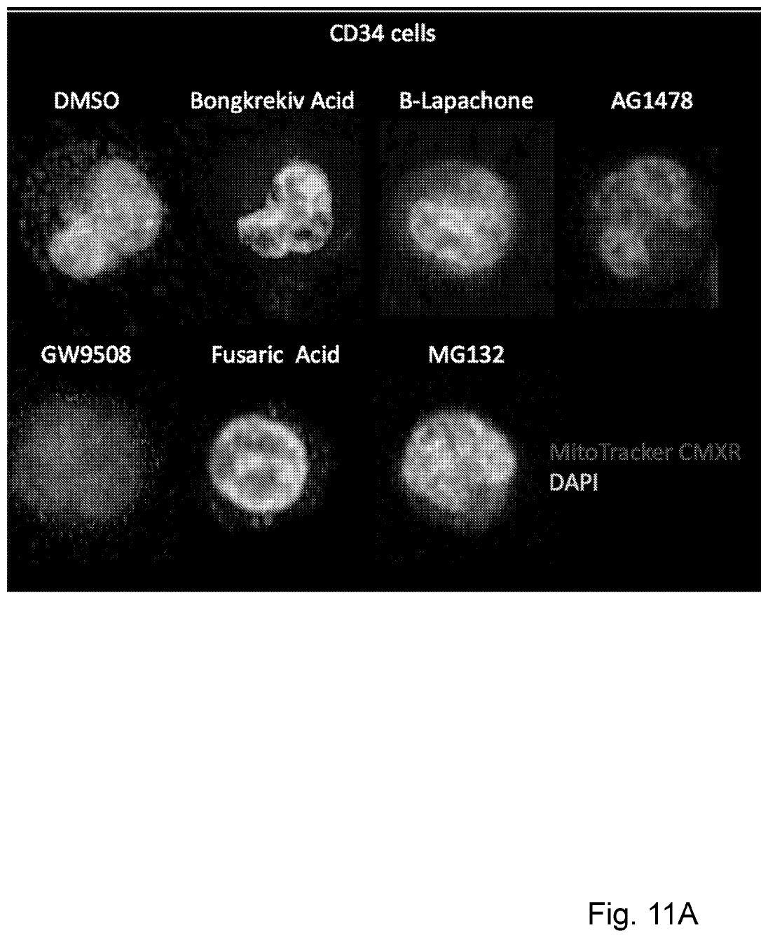

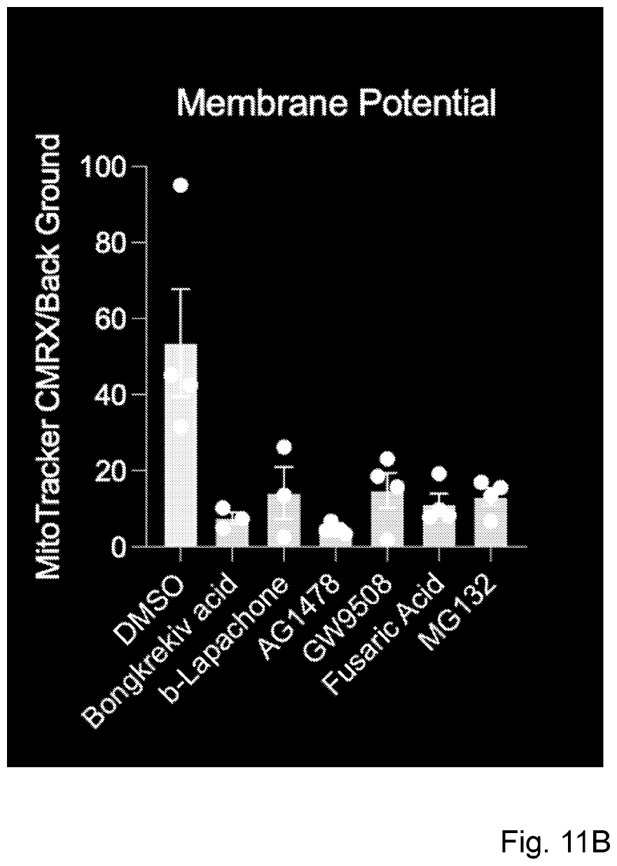

ROS+ compounds were associated with a “dooming” fate and altered the mitochondrial potential (FIGS. 10A-10B, 11A-11B). FIG. 10B shows the subset of FIG. 10 which is shaded.

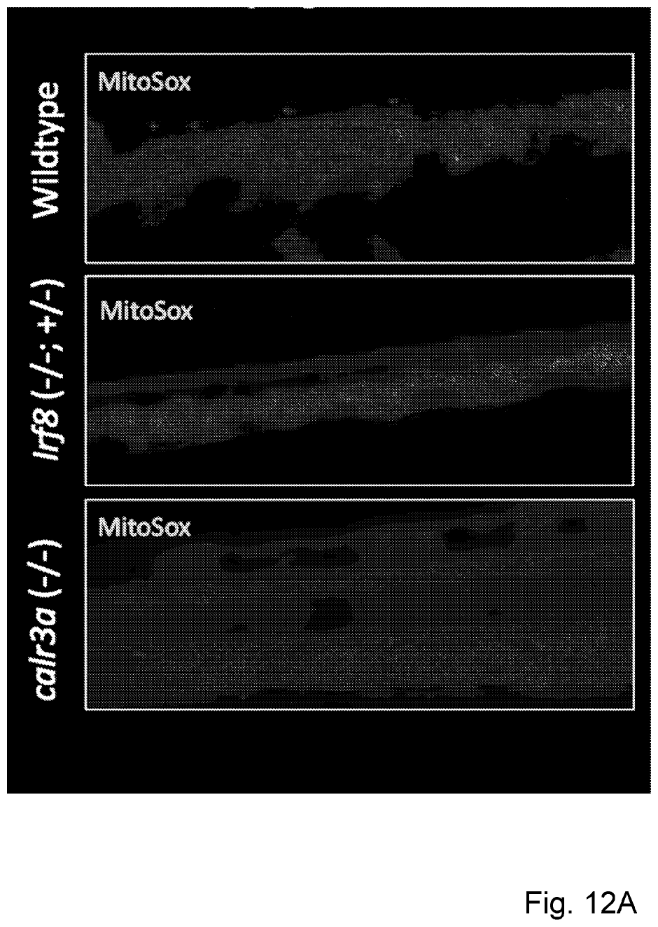

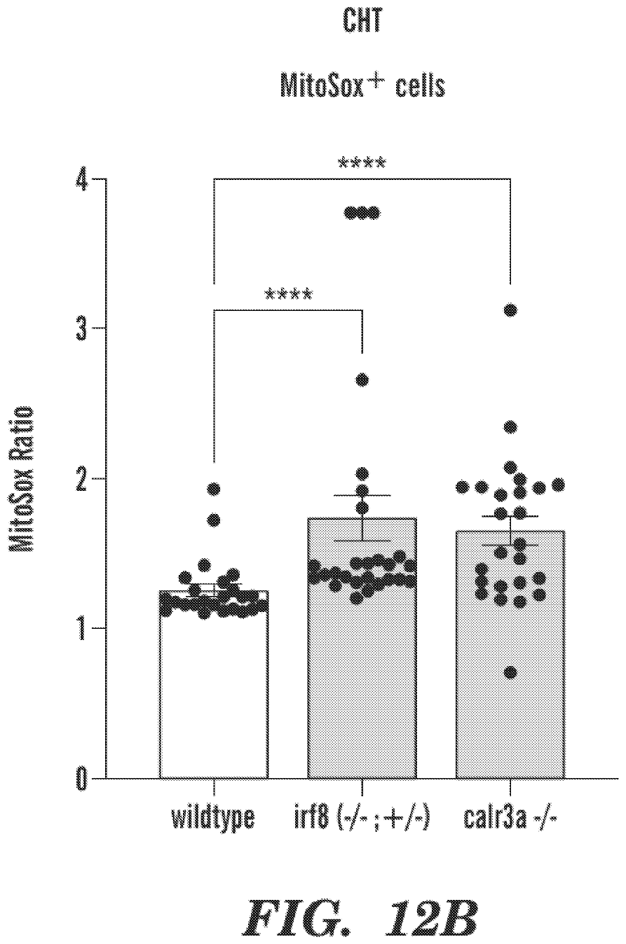

The absence of HSPC-macrophage interaction caused the accumulation of mitochondrial superoxide (FIGS. 12A-12B).

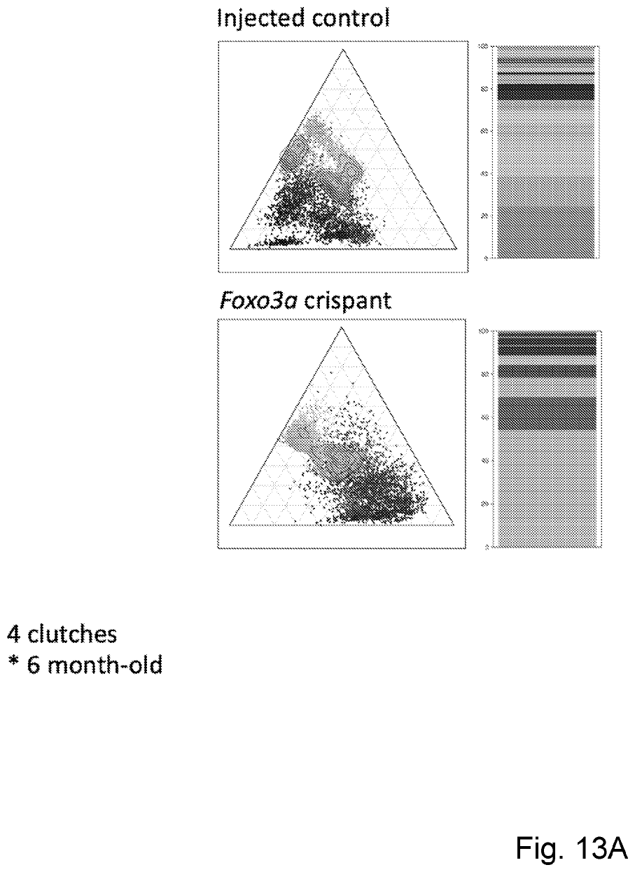

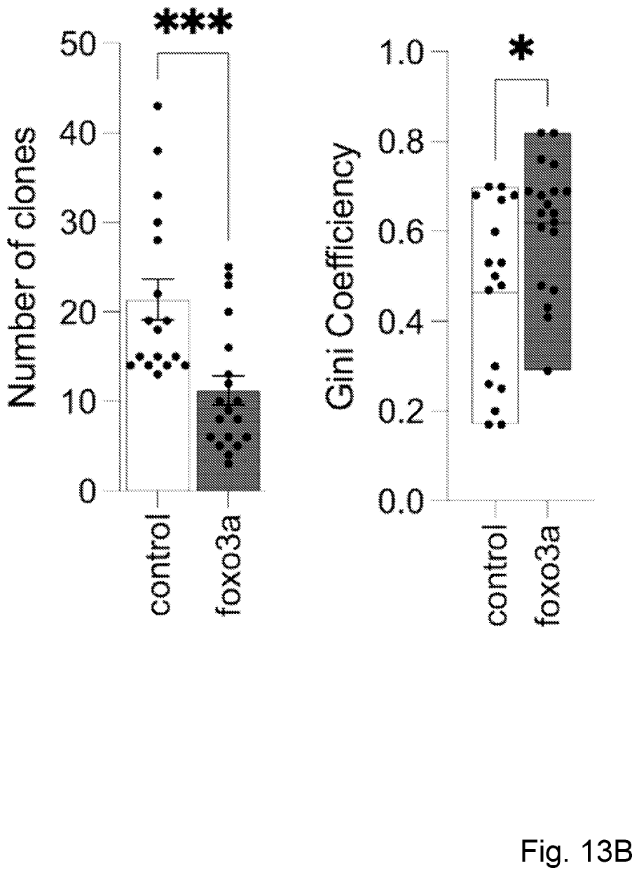

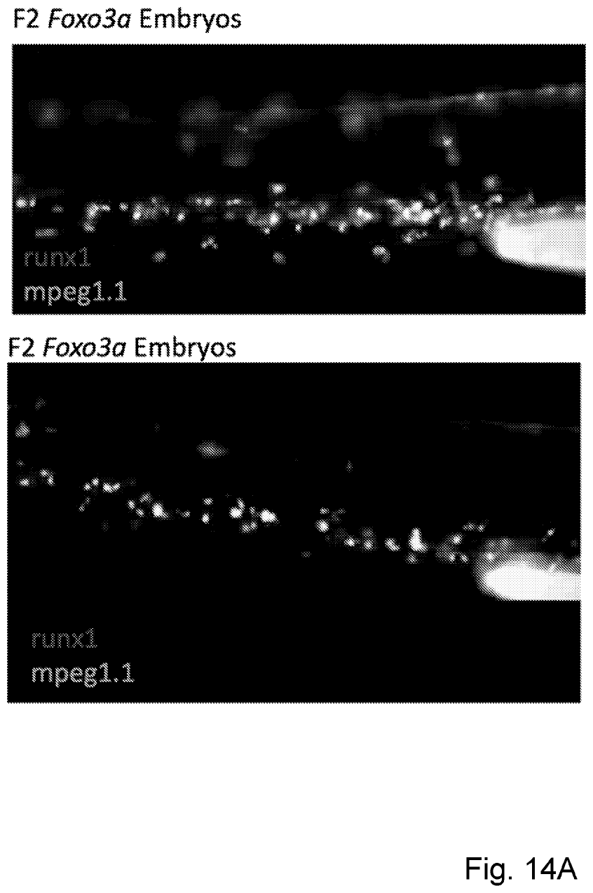

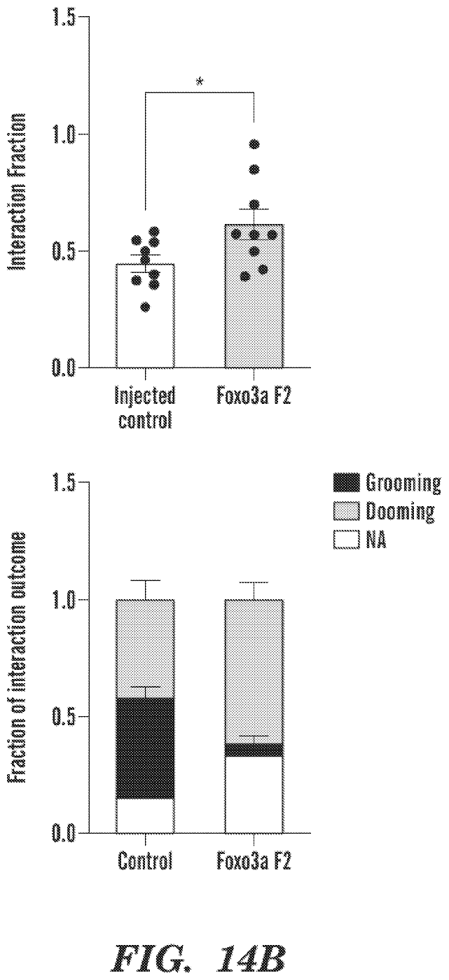

ROS levels altered stem cell clonality. For example Foxo3a (F0) CRIPSANTS had lower clonal diversity (FIGS. 13A-13B). Foxo3a germlines reproduced the F0 phenotype (FIGS. 14A-14B).

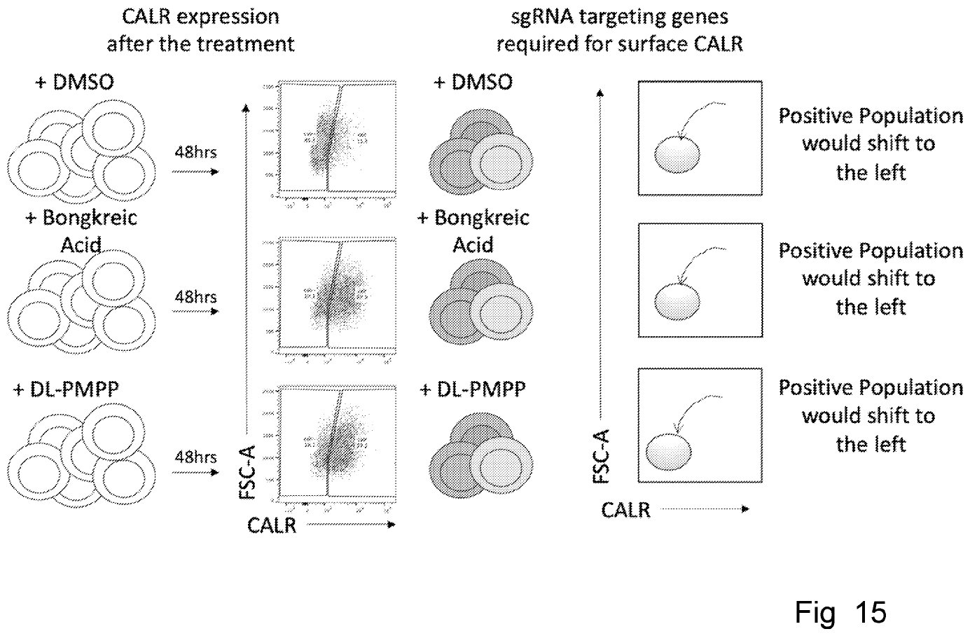

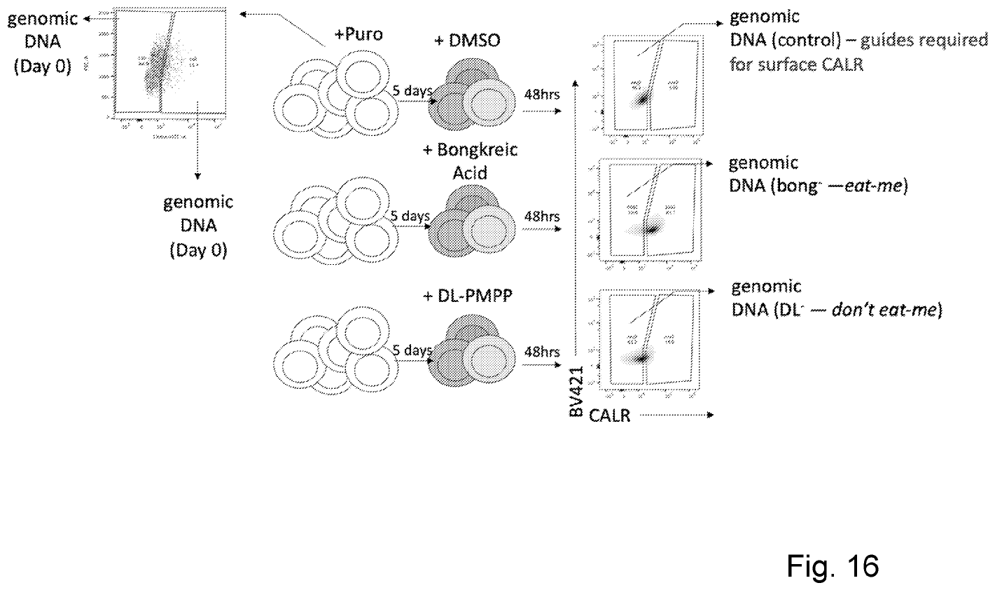

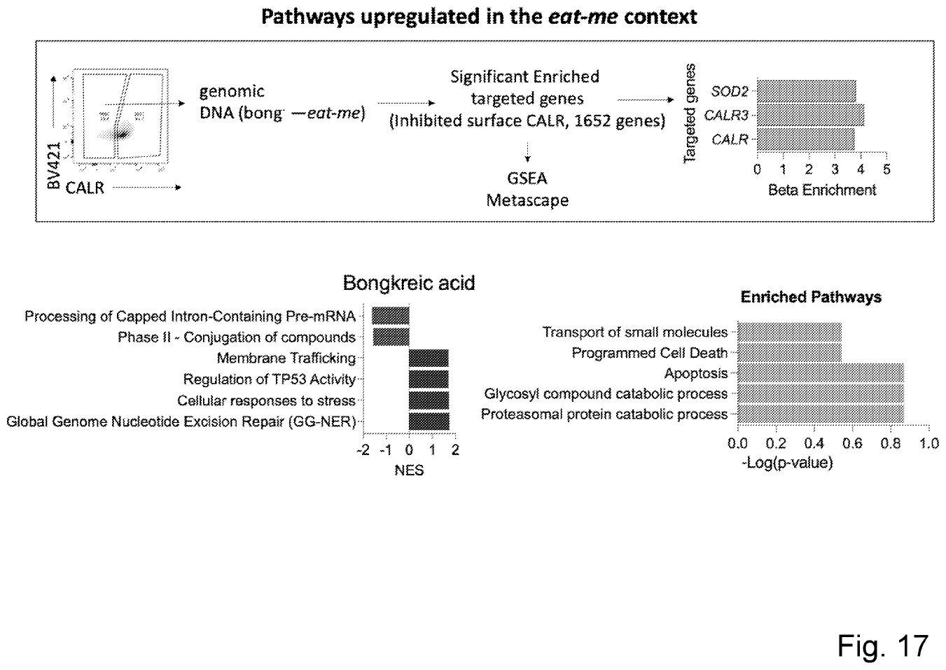

The experimental design for a CRISPR-Cas9 screen to decode the molecular cues regulating surface CALR is shown in FIGS. 15-16. The pathways upregulated in the eat-me context are shown in FIG. 17.

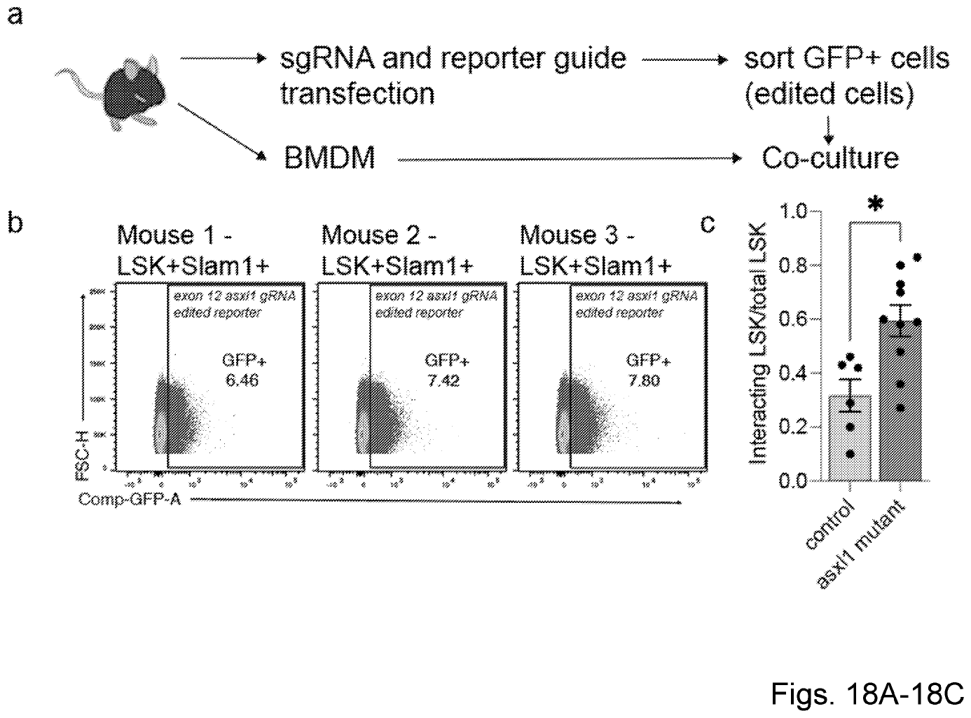

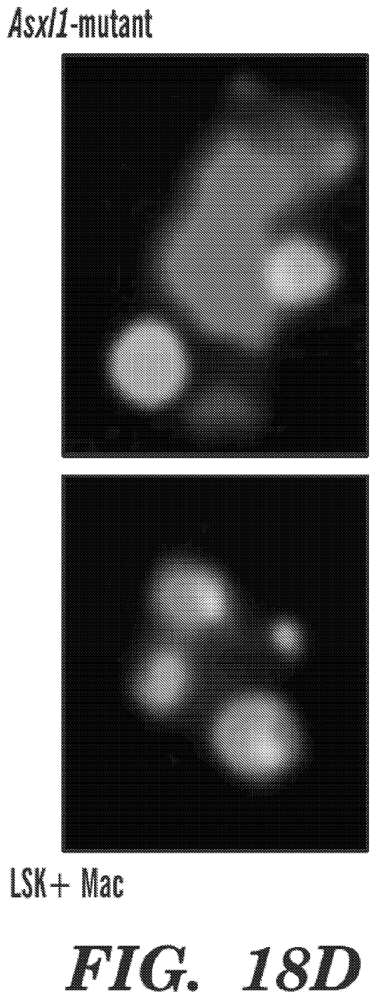

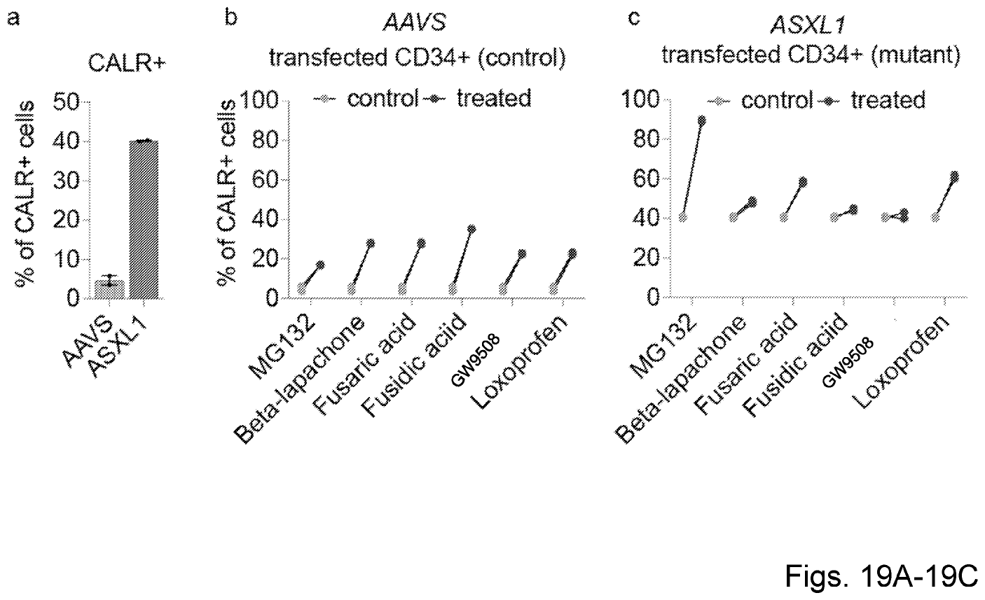

Asxl1-mutant progenitor cells showed higher engagement with macrophages (FIGS. 18A-18D). Asx1-mutant progenitor cells have higher surface CALR that can be modulated with the ROS/CALR inducers compounds (FIGS. 19A-19C).

1. A method of reducing hematopoiesis in a subject in need thereof, the method comprising administering at least one CALR agonist to the subject.

2. The method of claim 1, wherein the subject is a subject in need of treatment for clonal hematopoiesis, Clonal Hematopoiesis of Indeterminate Potential (CHIP), myelodysplastic syndrome (MDS), or leukemia.

3. The method of any one of the preceding claims, wherein the hematopoiesis is pathological hematopoiesis.

4. The method of any one of the preceding claims, wherein the administration is oral or intravenous.

5. (canceled)

6. The method of claim 1, whereby mitochondrial stimulation produces ROS.

7. The method of claim 6, wherein mitochondrial stimulation comprises mitochondrial modulation.

8. The method of claim 1, wherein the at least one CALR agonist is at least one ROS− CALR agonist.

9. The method of claim 8, wherein the at least one ROS− CALR agonist is selected from the group consisting of: DL-threo-1-Phenyl-2-pahnitoylamino-3-morpholino-1-propanol; cambinol; ganciclovir; 8-bromo-GMP; arvanil; thiocitrulline; and isoguvacine.

10. The method of claim 1, wherein the at least one CALR agonist is at least one ROS+ CALR agonist.

11. The method of claim 10, wherein the at least one ROS+ CALR agonist is selected from the group consisting of: Ibudilast; zaprinast; FK-520; AG1478; bongkrekic acid; GW9508; fusidic acid; beta-lapachone; fusaric acid; fenspiride; zardaverine; docosahexaenoic acid, loxoprofen; AG1480; flufenamic acid; and MG-132.

12. The method of claim 1, the method further comprising administering to the subject at least one chemotherapeutic.

13. The method of claim 12, wherein the at least one chemotherapeutic is selected from 5-azacytidine; venetoclax; and guadecitabine.

14. A method of improving proliferation and/or survival of healthy hematopoietic stem cells, the method comprising contacting a population of hematopoietic stem cells (HSCs) with at least one CALR agonist.

15. The method of claim 14, wherein the at least one CALR agonist is at least one ROS-CALR agonist.

16. The method of claim 15, wherein the at least one ROS− CALR agonist is selected from the group consisting of: DL-threo-1-Phenyl-2-pahnitoylamino-3-morpholino-1-propanol; cambinol; ganciclovir; 8-bromo-GMP; arvanil; thiocitrulline; and isoguvacine.

17. The method of claim 14, wherein the contacting occurs ex vivo.

18. The method of claim 17, wherein the population of HSCs and/or the progeny of the population of HSCs is subsequently administered to a subject.

19. The method of claim 14, wherein the contacting comprises administering the at least one CALR agonist to a subject in need of improved proliferation and/or survival of healthy hematopoietic stem cells.

20. The method of claim 19, wherein the subject in need of improved proliferation and/or survival of healthy hematopoietic stem cells is a subject in need of or receiving a transplant or adoptive cell therapy.

21. The method of claim 18, wherein the administration is oral or intravenous.

22.-44. (canceled)
